(19) United States
(12) Patent Application Publication
(10) Pub. No.: US 2026/0369379 A1
(43) Pub. Date: Nov. 19, 2026

(54) SYSTEMS AND METHODS FOR PATIENT-TREATMENT ASSOCIATION

(71) Applicant: 4U Medical Designs LLC, Lindenwood, NJ (US)

(72) Inventor: Nicholas Nastasi, Pine Hill, NJ (US)

(73) Assignee: 4U Medical Designs LLC, Lindenwood, NJ (US)

(21) Appl. No.: 19/681,236

(22) Filed: May 19, 2026

Related U.S. Application Data
(60) Provisional application No. 63/808,193, filed on May 19, 2025.

(51) Int. Cl. G16H 20/17

(52) U.S. Cl. CPC G16H 20/17

(57) ABSTRACT

A system for a patient check-in at a medical treatment facility includes a user interface, a data interface, and a computer. A patient may interact with the user interface to provide patient identification during check-in for an appointment. The computer communicates, via the data interface, with one or more external data sources to obtain appointment-specific treatment information associated with the patient and the appointment. The computer generates a unique identifier based on the patient identification and the appointment-specific treatment information, and generates a physical identifier including the patient identification, the appointment-specific treatment information, and the unique identifier. The physical identifier may be provided to a medical professional and may be usable to confirm the patient identification and/or the appointment-specific treatment information at a point of treatment. The unique identifier provides a persistent linkage between the physical identifier and a corresponding digital treatment record associated with the patient and the appointment.

### CROSS-REFERENCE TO RELATED APPLICATIONS

This application claims priority to and all benefits of U.S. Provisional Patent Application 63/808,193 filed on May 19, 2025, which is incorporated herein.

### BACKGROUND

Healthcare workflows may involve associating a patient with appointment-specific treatment information during one or more stages of a clinical workflow. As examples, healthcare workflows may involve retrieval, review, preparation, dispensing, transport, storage, verification, and/or administration of medications, infusion therapies, biologic treatments, injectable treatments, procedures, and/or other treatments associated with a patient appointment. In some healthcare environments, appointment-specific treatment information may be maintained within one or more electronic systems while treatment administration activities occur in separate physical workflow environments.

In some examples, healthcare workflows may utilize patient identification procedures, treatment verification procedures, printed records, labels, tickets, wristbands, and/or other identifiers during patient treatment activities. In some examples, healthcare workflows may involve communication between electronic health record systems, scheduling systems, pharmacy systems, insurance systems, treatment management systems, and/or other healthcare information systems. Coordination of patient identification information, appointment information, and treatment information across multiple systems and workflow stages may present technical and operational challenges.

### BRIEF DESCRIPTION OF THE DRAWINGS

FIG. 1 is a perspective view of a system for a patient check-in at a medical treatment facility.

FIG. 2 is a block diagram of components of an example of the system.

FIG. 3 is a flowchart of an example method.

FIG. 4 is a flowchart of another example method.

FIG. 5 is an example of a physical identifier being applied to a medical item.

FIG. 6 is a schematic of a workflow.

### SUMMARY

A system and method are disclosed for a patient check-in at a medical treatment facility. The system includes a user interface (e.g., a touchscreen on a kiosk or on a handheld tablet), a data interface, and a computer. A patient may interact with the user interface to provide patient identification during check-in for an appointment. The computer communicates, via the data interface, with one or more external data sources (e.g., one or more remote servers) to obtain appointment-specific treatment information associated with the patient and the appointment. The computer generates a unique identifier based on the patient identification and the appointment-specific treatment information, and generates a physical identifier including the patient identification, the appointment-specific treatment information, and the unique identifier. As examples, the physical identifier may include a printed label, sticker, wristband, ticket, tag, or card. In some examples, the computer may provide instructions to a printer to print at least a portion of the patient identification, the appointment-specific treatment information, and/or the unique identifier on the physical identifier. In some examples, the physical identifier may include a machine-readable code (e.g., a barcode or QR code) encoding at least a portion of the patient identification, the appointment-specific treatment information, and/or the unique identifier. The physical identifier may be provided to a medical professional (e.g., a doctor, physician assistant, nurse, pharmacist, or technician) and may be usable to confirm the patient identification and/or the appointment-specific treatment information during a treatment workflow and/or at a point of treatment. In some examples, the physical identifier may be associated with a medical item (e.g., an intravenous bag, syringe, medication container, or instrument) corresponding to the appointment-specific treatment. The unique identifier may be unique to the appointment and/or valid only for the appointment associated with the patient check-in.

In some examples, the system and method may improve identification of a patient and corresponding appointment-specific treatment information during a treatment workflow at a medical treatment facility. In some examples, the system may reduce reliance on manual identification and/or verification procedures performed by medical personnel. In some examples, the system may automatically retrieve appointment-specific treatment information associated with a patient during patient check-in and may generate a physical identifier corresponding to the patient and the appointment-specific treatment information. The physical identifier may be usable during one or more stages of a treatment workflow to facilitate association of the patient with a corresponding treatment and/or medical item. In some examples, the physical identifier may facilitate closed-loop verification at multiple stages of the clinical workflow, including preparation, dispensing, and/or administration of the treatment. In some examples, the physical identifier may facilitate verification that a correct patient is associated with a correct treatment and/or medical item during one or more stages of the clinical workflow. In some examples, the system may reduce a likelihood of treatment errors associated with incorrect patient identification, incorrect treatment identification, and/or incorrect association of a treatment with a patient. In some examples, the system may standardize treatment identification procedures across different medical treatment facilities and/or different treatment workflows. In some examples, the unique identifier may provide a linkage between the physical identifier and corresponding digital treatment information associated with the patient and the appointment.

In some examples, the user interface is accessible by a patient during a patient check-in associated with an appointment. The data interface is configured to communicate with an external data source to obtain the appointment-specific treatment information. The computer is programmed to receive patient identification from the user interface during the patient check-in for the appointment. The computer is programmed to transmit, via the data interface, a request for appointment-specific treatment information associated with the patient based on the patient identification. The computer is programmed to receive, via the data interface, the appointment-specific treatment information. The computer is programmed to generate a unique identifier based on the patient identification and the appointment-specific treatment information. The computer is programmed to generate, in response to receiving the appointment-specific treatment information during the patient check-in, a physical identifier including the patient identification, the appointment-specific treatment information, and the unique identifier.

In some examples, a method includes receiving patient identification via the user interface during the patient check-in associated with the appointment. The method includes transmitting, via the data interface, a request for appointment-specific treatment information associated with the patient based on the patient identification. The method includes receiving, via the data interface, the appointment-specific treatment information. The method includes generating a unique identifier based on the patient identification and the appointment-specific treatment information. The method includes generating, in response to receiving the appointment-specific treatment information during the patient check-in, a physical identifier including the patient identification, the appointment-specific treatment information, and the unique identifier.

### DETAILED DESCRIPTION

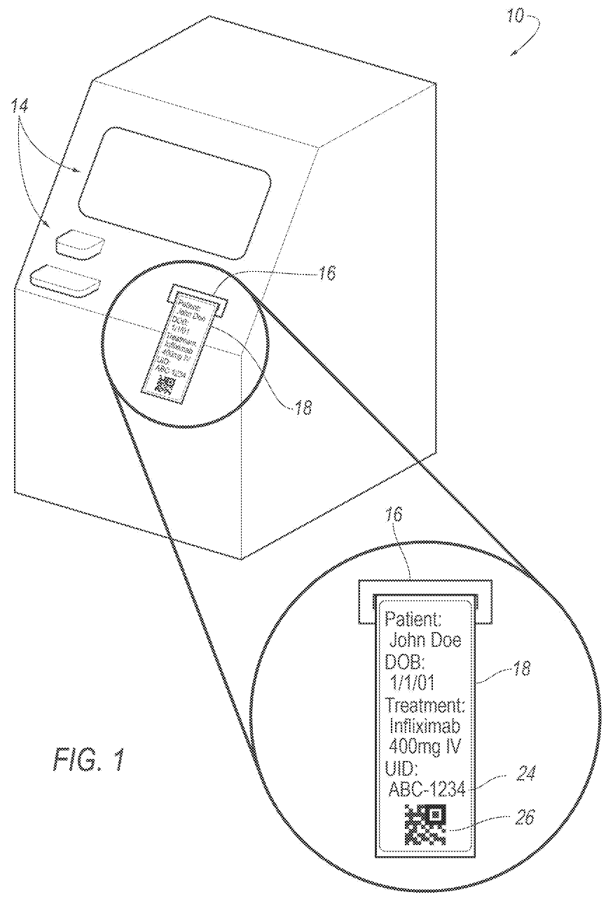

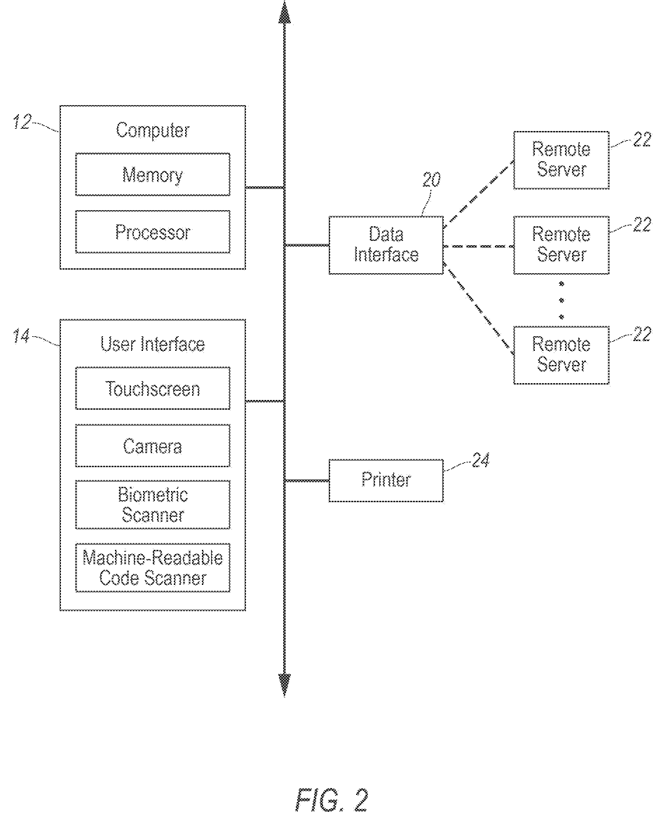

With reference to the Figures, wherein like numerals indicate like parts throughout the several views, a system 10 for self-service patient check-in at a medical treatment facility is generally shown. An example of the system 10 is shown in FIG. 1. FIG. 2 is a block diagram of components of an example of the system 10. The system 10 includes a user interface 14, a data interface 20, a printer 16, and a computer 12 in communication with the user interface 14, the data interface 20, and the printer 16. In some examples, the data interface 20 communicates with one or more external data sources 22 over a wired and/or wireless network.

The system 10 may be in the form of a kiosk (as shown in FIG. 1), a tablet, a laptop, a distributed computing system accessible via a network (e.g., a local and/or wide-area network), and/or another computing arrangement. As examples, the user interface 14 may communicate with a remote server, cloud computing platform, electronic health record system, and/or centralized application server over the network. In some examples, multiple kiosks, tablets, laptops, printers, and/or servers may communicate with one another over the network to perform one or more operations described herein.

As an example, the system 10 may be located at a medical treatment facility. Examples of medical treatment facilities include a hospital, clinic, physician office, urgent care facility, outpatient treatment center, infusion center, imaging center, laboratory, pharmacy, ambulatory care facility, surgical center, rehabilitation facility, and/or another healthcare facility. The system 10 may be positioned within a lobby, waiting area, check-in area, treatment area, hallway, patient room, nursing station, pharmacy area, and/or another location within the medical treatment facility. In some examples, the system 10 may be designed and/or positioned to reduce the likelihood of visibility of patient information by people other than the patient. As examples, the system 10 may include a privacy screen, side panels, directional display positioning, screen dimming, limited viewing angles, and/or physical spacing from adjacent patients and/or devices. In some examples, the system 10 may selectively display patient information during portions of the patient check-in process to reduce visibility of patient information to other people within the medical treatment facility.

The user interface 14 is accessible by a patient during a patient check-in associated with an appointment. The user interface 14 allows the patient to provide information to the computer 12 during the patient check-in. As examples, the patient may provide patient identification, appointment information, insurance information, demographic information, contact information, treatment information, and/or confirmation information via the user interface 14. In some examples, the user interface 14 may present prompts, instructions, confirmation screens, treatment information, and/or status information to the patient during the patient check-in process.

In some examples, the user interface 14 may include one or more modalities of input. As examples, the user interface 14 may include a touchscreen, a scanner, a camera, a keyboard, a microphone, a speaker, a display, and/or another input device. In some examples, a touchscreen (e.g., as shown in FIG. 1) may present prompts and/or selectable interface elements that allow the patient to input patient identification and/or other information associated with the appointment. In some examples, a scanner (shown in FIG. 1) may scan a patient identification card, insurance card, driver license, barcode, QR code, patient biometrics, and/or another machine-readable identifier associated with the patient and/or the appointment. In some examples, a camera may capture an image of the patient, a patient identification document, an insurance card, a barcode, a QR code, patient biometrics, and/or another identifier associated with the patient and/or the appointment. In some examples, information obtained via the touchscreen, scanner, and/or camera may be transmitted to the computer 12 for identification of the patient and retrieval of the appointment-specific treatment information.

The data interface 20 is configured to communicate with at least one external data source 22 to obtain appointment-specific treatment information. In some examples, the data interface 20 may communicate with a plurality of external data sources 22. As examples, the external data sources 22 may include an electronic health record system, a scheduling system, an insurance system, a pharmacy system, a laboratory system, a billing system, a treatment management system, a remote server, a cloud computing platform, and/or another local and/or remote computing system. In some examples, the external data sources 22 may store electronic health records (EHR) associated with the patient. Examples of EHR include patient demographic information, appointment information, medication information, prescription information, treatment information, procedure information, infusion information, dose information, route of administration information, physician information, insurance information, authorization information, laboratory results, allergy information, contraindication information, patient history information, diagnosis information, treatment preparation instructions, and/or other healthcare-related information associated with the patient and/or the appointment. In some examples, the data interface 20 may communicate with the external data sources 22 over a wired and/or wireless network using one or more communication protocols, application programming interfaces (APIs), web services, database queries, Health Level Seven (HL7) communications, Fast Healthcare Interoperability Resources (FHIR) communications, and/or other communication techniques.

In some examples, the appointment-specific treatment information may include at least a portion of the EHR associated with a current appointment of the patient and may correspond to a treatment to be administered during the appointment. In some examples, the computer 12 may combine information received from multiple external data sources 22 to generate the appointment-specific treatment information and/or a unique identifier 24 (described below).

In some examples, the external data source 22 may include one or more local and/or remote computing devices configured to store, process, transmit, and/or manage information associated with the patient and/or the appointment. As examples, the external data source 22 may include a server, database server, application server, cloud computing server, electronic health record (EHR) server, scheduling server, pharmacy server, insurance server, laboratory server, and/or another computing device configured to store and/or manage electronic health data. In some examples, the external data source 22 may be located within the medical treatment facility, at a remote data center, at a cloud computing environment, and/or at another local and/or remote location accessible via the network.

In some examples, the external data source 22 may execute one or more software applications configured to manage appointment information, treatment information, patient records, billing information, insurance information, medication information, and/or other healthcare-related information associated with the patient. In some examples, the external data source 22 may maintain electronic health records for a plurality of patients and may receive requests from the computer 12 for appointment-specific treatment information associated with a particular patient and/or appointment. In some examples, the external data source 22 may retrieve information from multiple databases and/or multiple healthcare management systems in response to a request received from the computer 12.

In some examples, the external data source 22 may transmit appointment-specific treatment information to the computer 12 in response to receiving patient identification and/or appointment information from the computer 12. In some examples, the appointment-specific treatment information transmitted by the external data source 22 may include treatment identification information, medication information, dose information, route of administration information, scheduling information, authorization information, allergy information, contraindication information, laboratory information, physician information, and/or other information associated with treatment of the patient during the appointment.

The printer 16 is configured to print information on the physical identifier 18 based on commands from the computer 12. In some examples, the physical identifier 18 may include a label, sticker, wristband, ticket, tag, card, printout, and/or another physical medium configured to present information associated with the patient and the appointment-specific treatment information to the medical provider. In some examples, the printer 16 may include a label printer, wristband printer, desktop printer, and/or another printing device configured to generate the physical identifier 18.

In some examples, the physical identifier 18 may include patient identification, appointment information, appointment-specific treatment information, and/or a unique identifier 24 generated based on the patient identification and the appointment-specific treatment information. As examples, the appointment-specific treatment information printed on the physical identifier 18 may include a medication name, treatment name, dose information, route of administration information, infusion information, physician information, scheduling information, treatment preparation instructions, and/or other information associated with the appointment. In some examples, the physical identifier 18 may include both human-readable information and machine-readable information (e.g., a machine-readable code 26). As examples, the machine-readable code 26 may include a barcode, QR code, alphanumeric code, encoded identifier, and/or another machine-readable code encoding at least a portion of the patient identification, the appointment-specific treatment information, and/or the unique identifier 24.

In some examples, the physical identifier 18 may be configured to be carried by the patient, presented to medical personnel, associated with a medical item, scanned by a scanner, visually inspected by medical personnel, and/or used during one or more stages of a treatment workflow. In some examples, the physical identifier 18 may provide a linkage between the patient, the appointment, the appointment-specific treatment information, and corresponding electronic health data associated with the patient (e.g., the EHR).

The computer 12 has a processor and memory storing instructions executable by the processor. In some examples, the computer 12 may include one or more processors, controllers, microprocessors, central processing units (CPUs), graphics processing units (GPUs), application-specific integrated circuits (ASICs), programmable logic devices, and/or other processing devices configured to execute instructions and/or process information. In some examples, the memory may include volatile memory, non-volatile memory, random-access memory (RAM), read-only memory (ROM), flash memory, solid-state memory, magnetic storage, optical storage, cloud-based storage, and/or another storage medium configured to store instructions and/or data.

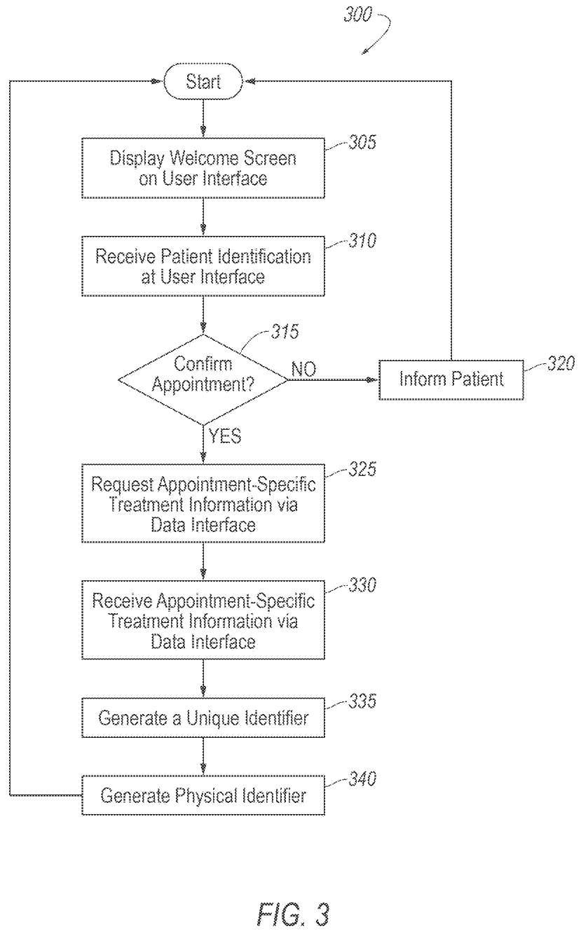

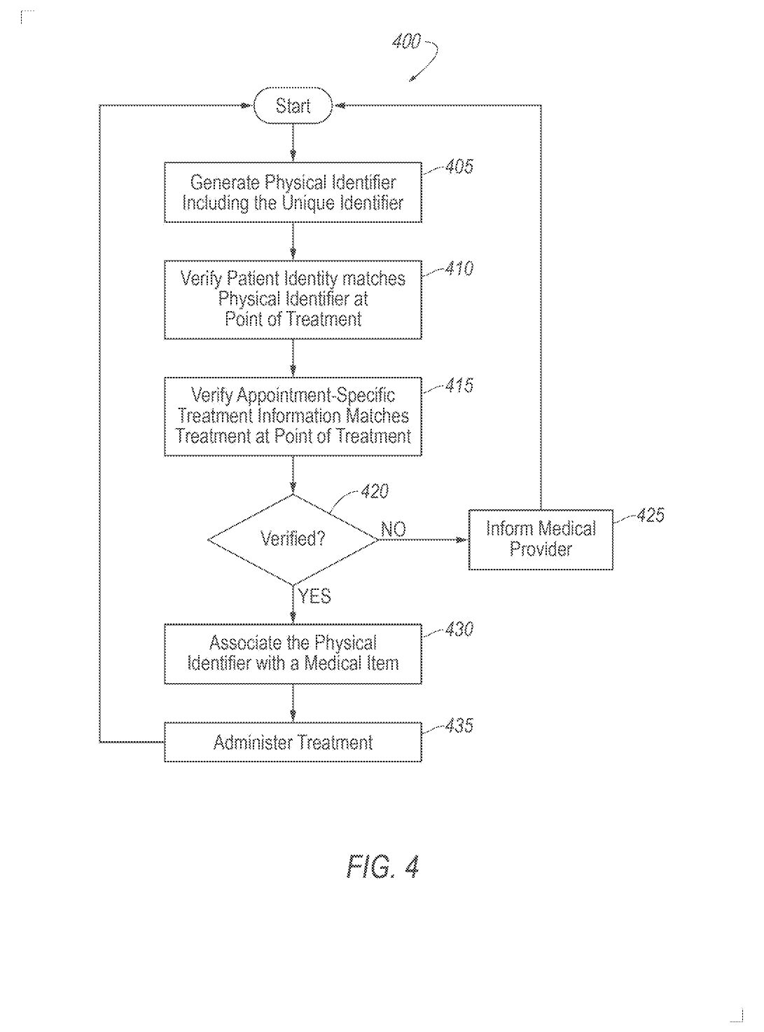

In some examples, the instructions stored in the memory may cause the processor to perform one or more operations described herein, including at least portions of the example methods 300 and 400 shown in FIGS. 3 and 4, respectively. As examples, the processor may execute instructions to receive patient identification from the user interface 14, communicate with one or more external data sources 22 via the data interface 20, retrieve appointment-specific treatment information associated with a patient and an appointment, generate a unique identifier 24 based on the patient identification and the appointment-specific treatment information, generate a physical identifier 18, transmit one or more commands (e.g., printing instructions) to the printer 16, encode information into a machine-readable code 26, validate appointment-specific treatment information, facilitate verification during a treatment workflow, record verification information, and/or perform one or more other operations described herein.

In some examples, the computer 12 may be implemented as a local computing device, a remote computing device, a server, a cloud computing device, a distributed computing system, and/or another computing arrangement. In some examples, one or more operations of the computer 12 may be distributed across multiple computing devices communicating over a network. In some examples, the instructions may be implemented as software, firmware, hardware, and/or combinations thereof.

The computer 12 is programmed to receive patient identification from the user interface 14 during the patient check-in for the appointment. In some examples, the patient identification may include a patient name, date of birth, patient number, account number, insurance information, biometric information, appointment confirmation information, and/or another identifier associated with the patient. In some examples, the patient identification may be manually entered by the patient via the user interface 14. In some examples, the patient identification may be automatically obtained by scanning and/or imaging a patient identification card, insurance card, driver license, barcode, QR code, biometric identifier, and/or another identifier associated with the patient.

In some examples, the computer 12 may analyze information received from the user interface 14 to identify the patient and/or identify an appointment associated with the patient. In some examples, the computer 12 may compare the received patient identification to information stored in one or more external data sources 22 to identify the patient and/or retrieve appointment information associated with the patient. In some examples, the computer 12 may determine whether multiple appointments are associated with the patient and may prompt the patient to select an appointment associated with the patient check-in. In some examples, the computer 12 may determine whether the patient identification corresponds to a scheduled appointment within a predetermined time period and may initiate retrieval of appointment-specific treatment information associated with the appointment.

In some examples, the computer 12 may present prompts, instructions, confirmation requests, and/or error messages via the user interface 14 during identification of the patient. As examples, the computer 12 may request confirmation of patient information, request additional identifying information, request confirmation of an appointment, and/or indicate that patient identification could not be verified. In some examples, the computer 12 may receive confirmation information from the patient indicating that the patient identification and/or appointment information is correct prior to retrieval of the appointment-specific treatment information.

In some examples, the computer 12 may transmit, via the data interface 20, a request for appointment-specific treatment information associated with the patient based on the patient identification received during the patient check-in. In some examples, the request may include the patient identification, appointment information, scheduling information, insurance information, and/or another identifier associated with the patient and/or the appointment. In some examples, the request transmitted by the computer 12 may cause the external data source 22 to identify an appointment associated with the patient and retrieve appointment-specific treatment information corresponding to the appointment.

In some examples, the appointment-specific treatment information may include at least one medication, dose information, and/or administration route information associated with the appointment. As examples, the medication may include an infusion medication, biologic medication, chemotherapy medication, injectable medication, prescription medication, and/or another treatment-related medication associated with the patient and/or the appointment. In some examples, the dose information may include a dosage amount, infusion quantity, concentration, preparation parameter, administration timing parameter, and/or another parameter associated with administration of the medication. In some examples, the administration route information may include intravenous administration information, intramuscular administration information, subcutaneous administration information, oral administration information, infusion administration information, and/or another administration route associated with treatment of the patient.

In some examples, the computer 12 may validate the appointment-specific treatment information against a patient profile associated with the patient (e.g., on an external data source 22) prior to generation of the unique identifier 24. As examples, the computer 12 may determine whether the appointment-specific treatment information corresponds to demographic information, allergy information, contraindication information, authorization information, physician information, scheduling information, insurance information, and/or other information associated with the patient and the appointment. In some examples, the computer 12 may associate the appointment-specific treatment information with the current patient encounter and may generate the unique identifier 24 linking the patient identification, the appointment, and the appointment-specific treatment information associated with the patient encounter. In some examples, generation of the unique identifier 24 may occur in response to validation of the appointment-specific treatment information associated with the patient and the appointment.

In some examples, the computer 12 may receive, via the data interface 20, the appointment-specific treatment information from one or more external data sources 22 in response to transmission of the request for the appointment-specific treatment information. In some examples, the appointment-specific treatment information received via the data interface 20 may correspond to a current appointment associated with the patient identified during the patient check-in. In some examples, the computer 12 may analyze, organize, store, and/or process the received appointment-specific treatment information prior to generation of the unique identifier 24 and/or the physical identifier 18. In some examples, the computer 12 may combine appointment-specific treatment information received from multiple external data sources 22 to generate a combined set of appointment-specific treatment information associated with the patient and the appointment.

As set forth above, in some examples, the computer 12 may generate a unique identifier 24 based on the patient identification and the appointment-specific treatment information. In some examples, the unique identifier 24 may be generated after receipt of the appointment-specific treatment information associated with the patient and the appointment. The unique identifier 24 uniquely corresponds to the patient and the appointment associated with the patient check-in. The unique identifier 24 provides a linkage between the patient identification, the appointment, the appointment-specific treatment information, the physical identifier 18, and/or corresponding electronic health data associated with the patient (e.g., the EHR).

In some examples, the unique identifier 24 may provide a persistent association between the physical identifier 18 and a corresponding digital treatment record associated with the patient and the appointment. In some examples, the unique identifier 24 may be usable to retrieve appointment-specific treatment information, electronic health data, treatment records, workflow information, verification records, and/or other information associated with the patient and the appointment from one or more external data sources 22. In some examples, the unique identifier 24 may facilitate retrieval of the corresponding digital treatment record during multiple stages of a clinical workflow without requiring repeated manual entry and/or re-identification of the patient. In some examples, the persistent association facilitated by the unique identifier 24 may support closed-loop verification, workflow traceability, audit tracking, and/or continuity of treatment information throughout the clinical workflow.

In some examples, the unique identifier 24 may be generated based on one or more of the patient identification, appointment information, treatment information, medication information, dose information, administration route information, scheduling information, timestamp information, random number information, sequential number information, encrypted information, encoded information, and/or another parameter associated with the patient and/or the appointment. In some examples, the unique identifier 24 may include a numeric identifier, alphanumeric identifier, barcode, QR code, encrypted identifier, encoded identifier, hash value, token, and/or another identifier configured to uniquely identify the appointment and corresponding appointment-specific treatment information.

The unique identifier 24 may be unique to the appointment associated with the patient check-in. The unique identifier 24 is generated for a single appointment associated with the patient and is different than other identifiers associated with other appointments of the patient. In some examples, the unique identifier 24 may correspond to a current treatment workflow associated with the appointment and may facilitate association of the patient with the appointment-specific treatment information during one or more stages of the treatment workflow.

In some examples, the unique identifier 24 may be valid only for the appointment associated with the patient check-in. In some examples, the computer 12 may determine that the unique identifier 24 is no longer valid after completion of the appointment, expiration of a predetermined time period, completion of administration of the appointment-specific treatment, termination of the appointment, and/or occurrence of another event associated with the appointment. In some examples, the computer 12 may prevent reuse of the unique identifier 24 for another appointment and/or another patient. In some examples, the unique identifier 24 may facilitate verification that the physical identifier 18 corresponds to the patient and the appointment-specific treatment information associated with the appointment.

In some examples, the computer 12 may generate, in response to receiving the appointment-specific treatment information during the patient check-in, a physical identifier 18 including the patient identification, the appointment-specific treatment information, and the unique identifier 24. In some examples, the computer 12 may generate the physical identifier 18 after identification of the patient and retrieval of the appointment-specific treatment information associated with the appointment. In some examples, the physical identifier 18 may correspond to a current appointment associated with the patient and may be generated during the patient check-in process. In some examples, the computer 12 may generate the physical identifier 18 based on a combination of the patient identification, the appointment-specific treatment information, and the unique identifier 24 such that the physical identifier 18 corresponds to the patient and the appointment-specific treatment information associated with the appointment.

In some examples, the physical identifier 18 may be configured to associate the patient with the appointment-specific treatment information within a clinical workflow. In some examples, the physical identifier 18 may be usable during one or more stages of the clinical workflow to facilitate association of the patient with a corresponding treatment, medication, procedure, and/or medical item associated with the appointment-specific treatment information. As examples, the physical identifier 18 may be used during preparation of a treatment, dispensing of a treatment, transport of a treatment, storage of a treatment, review of a treatment, verification of a treatment, and/or administration of a treatment associated with the patient. In some examples, the physical identifier 18 may facilitate closed-loop verification at multiple stages of the clinical workflow such that the patient identification, appointment-specific treatment information, and/or unique identifier 24 remain associated with one another during the clinical workflow.

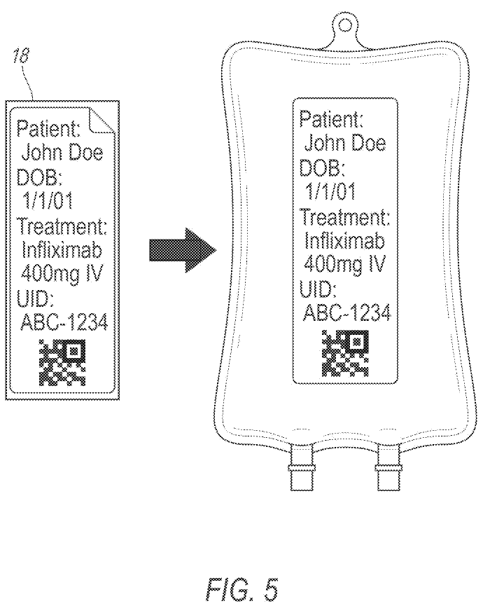

In some examples, the physical identifier 18 may include a human-readable representation of the appointment-specific treatment information, as shown, for example, in FIGS. 1 and 5. As examples, the human-readable representation may include a medication name, treatment name, dose information, administration route information, infusion information, physician information, scheduling information, warning information, preparation instructions, patient information, and/or other information associated with treatment of the patient during the appointment. In some examples, the human-readable representation may be printed as text, symbols, graphics, color coding, and/or another visually perceptible representation configured to be viewed by the patient and/or medical personnel during the clinical workflow.

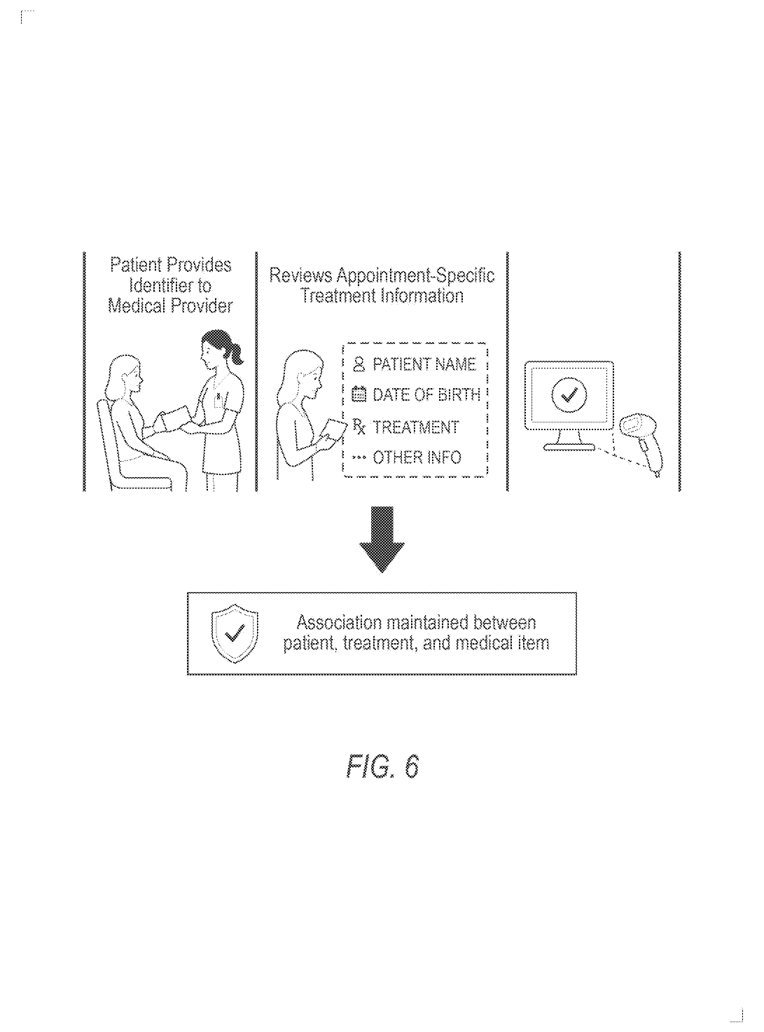

In some examples, the physical identifier 18 may include a machine-readable code 26 encoding at least the unique identifier 24, as shown, for example, in FIGS. 1 and 5. As examples, the machine-readable code 26 may include a barcode, QR code, matrix code, alphanumeric code, optical code, encoded image, and/or another machine-readable representation configured to encode information associated with the patient and the appointment. In some examples, the computer 12 may generate the machine-readable code 26 by encoding at least the unique identifier 24 into the machine-readable code 26 such that the machine-readable code 26 corresponds to the patient, the appointment, and/or the appointment-specific treatment information associated with the appointment. In some examples, the machine-readable code 26 may be scanned by a scanner (e.g., as shown in FIG. 6), camera, mobile computing device, pharmacy system, treatment management system, and/or another computing device during the clinical workflow to retrieve information associated with the appointment-specific treatment information and/or the patient.

In some examples, the machine-readable code 26 may encode at least the unique identifier 24 and at least a portion of the appointment-specific treatment information. As examples, the appointment-specific treatment information encoded within the machine-readable code 26 may include medication information, dose information, administration route information, treatment information, infusion information, scheduling information, and/or other information associated with treatment of the patient during the appointment. In some examples, encoding the appointment-specific treatment information and the unique identifier 24 into the machine-readable code 26 may facilitate machine-based retrieval, processing, comparison, verification, and/or validation of information associated with the patient and the appointment during one or more stages of the clinical workflow. In some examples, the machine-readable code 26 may facilitate comparison of the encoded appointment-specific treatment information to treatment information associated with a medical item, electronic health data, pharmacy information, and/or another source of treatment information during the clinical workflow. In some examples, the machine-readable code 26 may facilitate verification that the patient is associated with a correct treatment and/or medical item during preparation, dispensing, and/or administration of the treatment.

An example method 300 is shown in FIG. 3. The method 300 is performed by the computer 12. The computer 12 is programmed to perform the operations of the method 300. Use of “in response to” and “based on” herein indicates a causal relationship, not merely a temporal relationship.

With reference to block 305, the method 300 includes displaying a welcome screen on the user interface 14. In some examples, the welcome screen may be displayed by the computer 12 on a touchscreen and/or another display of the user interface 14 in response to initiation of the patient check-in process. In some examples, the welcome screen may include prompts, instructions, selectable interface elements, graphics, branding information, facility information, appointment information, privacy information, and/or other information associated with the patient check-in process. In some examples, the welcome screen may prompt the patient to provide patient identification and/or appointment information associated with an appointment.

In some examples, the welcome screen may include instructions directing the patient to interact with one or more modalities of input of the user interface 14. As examples, the welcome screen may instruct the patient to provide information using a touchscreen, scanner, camera, keyboard, microphone, and/or another input device of the user interface 14. In some examples, the welcome screen may prompt the patient to scan a patient identification card, insurance card, barcode, QR code, and/or another identifier associated with the patient and/or the appointment. A scanner is shown as one of the user interfaces 14 in FIG. 1, for example. In some examples, the welcome screen may prompt the patient to manually enter patient identification and/or appointment information via the touchscreen.

In some examples, the welcome screen may be configured to initiate one or more operations of the method 300 associated with identification of the patient and retrieval of appointment-specific treatment information. In some examples, the computer 12 may modify information displayed on the welcome screen based on information received from the patient, information associated with the appointment, information received from one or more external data sources 22, and/or another parameter associated with the patient check-in process.

With reference to block 310, the method 300 includes receiving patient identification via the user interface 14 during a patient check-in associated with the appointment. In some examples, the computer 12 may receive the patient identification in response to interaction of the patient with the user interface 14 during the patient check-in process. In some examples, the patient identification may include a patient name, date of birth, patient number, account number, insurance information, appointment confirmation information, biometric information, and/or another identifier associated with the patient. In some examples, the computer 12 may receive the patient identification via one or more input modalities of the user interface 14, including a touchscreen (e.g., as shown in FIG. 1), scanner (e.g., as shown in FIG. 1), camera, keyboard, microphone, and/or another input device.

In some examples, receiving the patient identification may include manually entering patient information via the touchscreen, scanning a patient identification card and/or insurance card, capturing an image of a patient identification document, scanning a barcode and/or QR code associated with the patient, and/or receiving biometric information associated with the patient. In some examples, the computer 12 may analyze the received patient identification to identify the patient and/or determine an appointment associated with the patient. In some examples, the computer 12 may compare the received patient identification to electronic health data stored in one or more external data sources 22 to identify the patient and retrieve appointment information associated with the patient. In some examples, the computer 12 may determine whether multiple appointments are associated with the patient and may prompt the patient to select an appointment associated with the patient check-in. In some examples, the computer 12 may determine that the patient identification corresponds to a scheduled appointment within a predetermined time period prior to retrieval of the appointment-specific treatment information.

With reference to block 315, the method 300 includes confirming the appointment associated with the patient identification received during the patient check-in. In some examples, the computer 12 may determine whether the patient identification corresponds to a scheduled appointment associated with the patient. In some examples, the computer 12 may confirm the appointment based on appointment information previously stored by the computer 12 and/or without accessing an external data source 22 via the data interface 20. In some examples, the computer 12 may request, receive, and/or access information from one or more external data sources 22 to determine whether an appointment associated with the patient is scheduled for a current date, time period, treatment location, medical treatment facility, medical provider, and/or treatment workflow. In some examples, the computer 12 may receive appointment information from one or more external data sources 22 and may determine appointment confirmation based on the received appointment information. In some examples, the computer 12 may receive a determination of appointment confirmation from one or more external data sources 22.

With reference to block 320, in the event that the appointment is not confirmed, the method 300 includes informing the patient. As examples, the computer 12 may determine that the patient has arrived on an incorrect date, at an incorrect time, at an incorrect treatment location, and/or without a scheduled appointment associated with the patient identification. In some examples, the computer 12 may inform the patient via the user interface 14 that an appointment could not be confirmed. As examples, the user interface 14 may display an error message, notification, instruction, appointment status message, scheduling message, and/or another indication that the appointment was not confirmed. In some examples, the computer 12 may prompt the patient to re-enter patient information, select another appointment, contact medical personnel, contact a scheduling department, and/or initiate another action associated with the patient check-in process.

With reference to block 325, the method 300 includes transmitting, via a data interface 20, a request for appointment-specific treatment information associated with the patient based on the patient identification. In some examples, the computer 12 may transmit the request to one or more external data sources 22 in response to receiving the patient identification during the patient check-in process. In some examples, the request may include the patient identification, appointment information, scheduling information, insurance information, demographic information, and/or another identifier associated with the patient and/or the appointment. In some examples, the request transmitted via the data interface 20 may cause one or more external data sources 22 to identify an appointment associated with the patient and retrieve appointment-specific treatment information corresponding to the appointment.

In some examples, the request may be transmitted to an electronic health record system, scheduling system, insurance system, pharmacy system, treatment management system, cloud computing platform, remote server, and/or another external data source 22 configured to store electronic health data associated with the patient. In some examples, the request may be transmitted over a wired and/or wireless network using one or more communication protocols, application programming interfaces (APIs), web services, database queries, Health Level Seven (HL7) communications, Fast Healthcare Interoperability Resources (FHIR) communications, and/or other communication techniques.

In some examples, the appointment-specific treatment information requested by the computer 12 may include medication information, treatment information, dose information, administration route information, infusion information, physician information, scheduling information, authorization information, allergy information, contraindication information, laboratory information, and/or other information associated with treatment of the patient during the appointment. In some examples, the request may cause retrieval of appointment-specific treatment information associated with a current appointment of the patient identified during the patient check-in.

With reference to block 330, the method 300 includes receiving, via the data interface 20, the appointment-specific treatment information. In some examples, the computer 12 may receive the appointment-specific treatment information from one or more external data sources 22 in response to transmission of the request for the appointment-specific treatment information. In some examples, the appointment-specific treatment information received via the data interface 20 may correspond to a current appointment associated with the patient identified during the patient check-in. In some examples, the appointment-specific treatment information may include medication information, treatment information, dose information, administration route information, infusion information, physician information, scheduling information, authorization information, allergy information, contraindication information, laboratory information, and/or other information associated with treatment of the patient during the appointment.

In some examples, receiving the appointment-specific treatment information includes receiving appointment-specific treatment information from a plurality of external data sources 22. As examples, the computer 12 may receive a first portion of the appointment-specific treatment information from an electronic health record system, a second portion of the appointment-specific treatment information from a scheduling system, a third portion of the appointment-specific treatment information from a pharmacy system, and/or a fourth portion of the appointment-specific treatment information from an insurance system. In some examples, the computer 12 may combine information received from the plurality of external data sources 22 to generate a combined set of appointment-specific treatment information associated with the patient and the appointment.

In some examples, the computer 12 may compare, organize, filter, validate, prioritize, and/or process the appointment-specific treatment information received from the plurality of external data sources 22. In some examples, the computer 12 may determine whether inconsistencies exist between information received from different external data sources 22. As examples, the computer 12 may determine whether scheduling information corresponds to treatment information, whether medication information corresponds to authorization information, and/or whether treatment information corresponds to patient allergy information and/or contraindication information.

With reference to block 335, the method 300 includes generating a unique identifier 24 based on the patient identification and the appointment-specific treatment information. In some examples, the method 300 includes generating the unique identifier 24 after receiving the appointment-specific treatment information associated with the patient and the appointment via the data interface 20. The unique identifier 24 uniquely corresponds to the patient and the appointment associated with the patient check-in. The unique identifier 24 may provide a linkage between the patient identification, the appointment, the appointment-specific treatment information, the physical identifier 18, and corresponding electronic health data associated with the patient, as described above.

In some examples, generating the unique identifier 24 includes combining appointment-specific treatment information obtained from the plurality of external data sources 22. As examples, the method 300 includes combining medication information received from a pharmacy system, appointment information received from a scheduling system, treatment information received from an electronic health record system, authorization information received from an insurance system, and/or other information received from one or more external data sources 22 to generate the unique identifier 24. In some examples, the unique identifier 24 may be generated based on relationships between multiple categories of appointment-specific treatment information associated with the patient and the appointment.

In some examples, as set forth above, the unique identifier 24 may be generated based on one or more of the patient identification, appointment information, medication information, treatment information, dose information, administration route information, infusion information, physician information, timestamp information, location information, random number information, sequential number information, encrypted information, encoded information, and/or another parameter associated with the patient and/or the appointment. In some examples, the unique identifier 24 may include a numeric identifier, alphanumeric identifier, barcode identifier, QR code identifier, token, encrypted identifier, encoded identifier, hash value, and/or another identifier configured to uniquely identify the appointment and corresponding appointment-specific treatment information.

The unique identifier 24 is unique to the appointment associated with the patient check-in and may differ from identifiers associated with other appointments of the patient. In some examples, the unique identifier 24 may be valid only for the appointment associated with the patient check-in. In some examples, the method 300 may include determining that the unique identifier 24 is no longer valid after completion of the appointment, expiration of a predetermined time period, completion of administration of the appointment-specific treatment, termination of the appointment, and/or occurrence of another event associated with the appointment.

The unique identifier 24 facilitates association of the patient with the appointment-specific treatment information during one or more stages of a clinical workflow. In some examples, the unique identifier 24 may facilitate closed-loop verification during preparation, dispensing, transport, storage, review, verification, and/or administration of a treatment associated with the patient. In some examples, the unique identifier 24 may facilitate comparison, validation, and/or verification of appointment-specific treatment information associated with the patient during one or more stages of the clinical workflow.

In some examples, generating the unique identifier 24 includes encoding at least a portion of the appointment-specific treatment information into the unique identifier 24. In such examples, the encoded unique identifier 24 may be digitally generated (i.e., digitally represented in the computer 12, digitally stored on the computer 12, etc.) As set forth below, the encoded unique identifier 24 may be physically generated on the physical identifier 18.

The method 300 includes generating, in response to receiving the appointment-specific treatment information during the patient check-in, a physical identifier 18 including the patient identification, the appointment-specific treatment information, and the unique identifier 24. In some examples, the method 300 includes generating the physical identifier 18 such that the physical identifier 18 encodes relationships between the patient identification, the appointment, and the appointment-specific treatment information associated with the appointment. As examples, the appointment-specific treatment information encoded within the physical identifier 18 may include medication information, treatment information, dose information, administration route information, infusion information, scheduling information, physician information, and/or other information associated with treatment of the patient during the appointment.

In some examples, generating the physical identifier 18 includes combining and encoding appointment-specific treatment information obtained from a plurality of external data sources 22. As examples, the method 300 may include encoding information obtained from an electronic health record system, scheduling system, pharmacy system, insurance system, laboratory system, and/or another external data source 22 into the physical identifier 18. In some examples, the physical identifier 18 may be generated such that the physical identifier 18 corresponds to a combined set of appointment-specific treatment information associated with the patient and the appointment.

The method may include encoding appointment-specific treatment information and/or the unique identifier 24 on the physical identifier 18. As an example, the method 300 may include generating a machine-readable code 26 encoding appointment-specific treatment information and/or the unique identifier 24. As set forth above, in such examples, the machine-readable code 26 may be digitally generated, and in such examples may be stored, e.g., by the computer 12. As set forth below, the machine-readable code 26 may be physically generated on the physical identifier 18. In some examples, because the unique identifier 24 encodes at least a portion of the appointment-specific treatment information, the machine-readable code 26 may indirectly encode the appointment-specific treatment information through the unique identifier 24. In some examples, the machine-readable code 26 may encode at least a portion of the appointment-specific treatment information separately from the unique identifier 24. As examples, the machine-readable code 26 may include a barcode, QR code, matrix code, optical code, encoded image, and/or another machine-readable representation configured to encode information associated with the patient and the appointment.

In some examples, encoding the appointment-specific treatment information into encoded form, e.g., the machine-readable code 26, may facilitate machine-based retrieval, comparison, verification, validation, and/or association of information associated with the patient and the appointment during one or more stages of a clinical workflow. In some examples, the encoded information may facilitate closed-loop verification during preparation, dispensing, transport, storage, review, verification, and/or administration of a treatment associated with the patient.

With reference to block 340, the method 300 includes generating the physical identifier 18. In some examples, generating the physical identifier 18 includes generating the physical identifier 18 in response to receiving the appointment-specific treatment information during the patient check-in associated with the appointment. In some examples, the physical identifier 18 includes the patient identification, the appointment-specific treatment information, and the unique identifier 24 associated with the patient and the appointment. In some examples, generating the physical identifier 18 includes generating a printed label, sticker, wristband, ticket, tag, card, and/or another physical medium configured to present information associated with the patient and the appointment-specific treatment information. Generating the physical identifier 18 may include encoding information, e.g., the appointment-specific treatment information and/or unique identifier 24, as described above.

In some examples, generating the physical identifier 18 includes generating both a human-readable representation and a machine-readable representation associated with the patient and the appointment. As examples, the human-readable representation may include a patient name, treatment information, medication information, dose information, administration route information, scheduling information, physician information, warning information, preparation instructions, and/or other information associated with treatment of the patient during the appointment. In some examples, the machine-readable representation may include a machine-readable code 26 such as a barcode, QR code, matrix code, optical code, encoded image, and/or another machine-readable representation configured to encode information associated with the patient and the appointment.

In some examples, generating the physical identifier 18 includes generating the physical identifier 18 such that the physical identifier 18 associates the patient with the appointment-specific treatment information during one or more stages of a clinical workflow. As examples, the physical identifier 18 may be associated with a medical item, carried by the patient, presented to medical personnel, scanned by a computing device, visually reviewed by medical personnel, and/or otherwise used during preparation, dispensing, transport, storage, review, verification, and/or administration of a treatment associated with the patient.

In some examples, generating the physical identifier 18 includes generating the physical identifier 18 based on appointment-specific treatment information obtained from a plurality of external data sources 22. In some examples, the physical identifier 18 may correspond to a combined set of appointment-specific treatment information associated with the patient and the appointment. In some examples, the physical identifier 18 may facilitate closed-loop verification of the patient identification and the appointment-specific treatment information during multiple stages of the clinical workflow.

An example method 400 is shown in FIG. 4. The method 400 includes generation of the physical identifier 18, as described above with respect to the method 300. In some examples, the method 400 may be performed during one or more stages of a clinical workflow associated with treatment of the patient. As examples, the method 400 may be performed during preparation of a treatment, dispensing of a treatment, transport of a treatment, storage of a treatment, review of a treatment, verification of a treatment, and/or administration of a treatment associated with the patient. In some examples, the method 400 may utilize the physical identifier 18, the unique identifier 24, and/or the machine-readable code 26 to facilitate association of the patient with appointment-specific treatment information during the clinical workflow. In some examples, the method 400 may facilitate closed-loop verification of patient identification and appointment-specific treatment information during one or more stages of the clinical workflow prior to administration of treatment to the patient.

In block 405, the method 400 includes generating the physical identifier 18 including the unique identifier 24. The steps of method 300 may be incorporated into block 405. The physical identifier 18 generated at block 405 may be associated with the patient, carried by the patient, associated with a medical item, presented to medical personnel, scanned by a computing device, and/or otherwise utilized during one or more stages of the clinical workflow associated with treatment of the patient. In some examples, the physical identifier 18 generated at block 405 may facilitate maintenance of an association between the patient and the appointment-specific treatment information during subsequent stages of the clinical workflow.

With reference to blocks 410 and 415, the method 400 includes verifying, at a point of treatment, that the patient identification and/or the appointment-specific treatment information correspond to the physical identifier 18 prior to administration of the appointment-specific treatment. In some examples, the method 400 includes performing verification of the patient identification and verification of the appointment-specific treatment information separately. In some examples, the method 400 includes performing verification of the patient identification and verification of the appointment-specific treatment information simultaneously. In some examples, the method 400 includes performing one or more verification operations using information presented on, encoded within, and/or otherwise associated with the physical identifier 18. An example workflow of the verification is shown in FIG. 6.

In some examples, the method 400 includes performing the verification of the identity and/or the treatment information during one or more stages of a clinical workflow associated with treatment of the patient. As examples, the verification may occur during preparation of a treatment, dispensing of a treatment, transport of a treatment, storage of a treatment, review of a treatment, and/or administration of a treatment associated with the patient. In some examples, the method 400 includes performing one or more verification operations prior to administration of the appointment-specific treatment to the patient.

With reference to block 410, the method 400 includes receiving verification information associated with the patient prior to administration of the appointment-specific treatment. In some examples, the verification information may include the same and/or similar information used during the patient check-in process described above. As examples, the verification information may include a patient name, date of birth, patient number, account number, insurance information, appointment confirmation information, biometric information, and/or another identifier associated with the patient.

In some examples, block 410 includes receiving the verification information from the patient at a point of treatment. As examples, the patient may verbally provide identifying information, manually enter identifying information into a computing device, provide a patient identification card, provide an insurance card, provide a driver license and/or another identification document, and/or provide biometric information associated with the patient. In some examples, biometric information may include fingerprint information, facial recognition information, retinal information, voice information, and/or another biometric identifier associated with the patient.

In some examples, block 410 includes comparing the verification information received from the patient to patient identification associated with the physical identifier 18. In some examples, the method 400 includes determining whether the verification information received from the patient corresponds to patient identification presented on, encoded within, and/or otherwise associated with the physical identifier 18. In some examples, the method 400 includes verifying the identity of the patient in response to determining correspondence between the verification information and the patient identification associated with the physical identifier 18 prior to administration of the appointment-specific treatment.

With reference to block 415, in some examples, the method 400 includes comparing the appointment-specific treatment information associated with the patient to appointment-specific treatment information presented on and/or encoded within the physical identifier 18. As examples, the appointment-specific treatment information may include medication information, treatment information, dose information, administration route information, infusion information, physician information, scheduling information, and/or other information associated with treatment of the patient during the appointment.

In some examples, the method 400 includes visually comparing appointment-specific treatment information displayed on the physical identifier 18 to a treatment prepared for administration to the patient, as shown, for example, in FIG. 6. As examples, a medical provider may visually compare a medication name, treatment name, dose information, administration route information, infusion information, physician information, scheduling information, and/or other appointment-specific treatment information displayed on the physical identifier 18 to corresponding information associated with the treatment prepared for administration to the patient.

In some examples, the method 400 includes scanning a machine-readable code 26 associated with the physical identifier 18 to retrieve appointment-specific treatment information associated with the patient and the appointment, as shown, for example, in FIG. 6. As described above, the machine-readable code 26 may include a barcode, QR code, matrix code, optical code, encoded image, and/or another machine-readable representation configured to encode the unique identifier 24 and/or appointment-specific treatment information associated with the patient and the appointment.

In some examples, the method 400 includes accessing one or more external data sources 22 in response to scanning the machine-readable code 26. In some examples, the method 400 includes transmitting, via the data interface 20, a request for appointment-specific treatment information associated with the patient based on information obtained from the machine-readable code 26 and/or the unique identifier 24 encoded within the machine-readable code 26. In some examples, the method 400 includes receiving appointment-specific treatment information from one or more external data sources 22 in a manner similar to the retrieval of appointment-specific treatment information during the patient check-in process described above.

In some examples, the method 400 includes comparing the appointment-specific treatment information associated with the physical identifier 18 to stored treatment data, electronic health data, pharmacy information, treatment preparation information, and/or other information associated with treatment prepared for administration to the patient. In some examples, the method 400 includes determining whether the appointment-specific treatment information associated with the physical identifier 18 corresponds to the treatment prepared for administration to the patient prior to administration of the appointment-specific treatment.

In response to verification of patient identity and/or appointment-specific treatment information in blocks 410 and 415, in some examples, the method 400 includes associating the physical identifier 18 with a medical item to be used in administration of the appointment-specific treatment, as shown in block 420. As examples, the medical item may include an intravenous bag, syringe, medication container, injectable medication device, infusion device, instrument, specimen container, treatment kit, and/or another medical item associated with treatment of the patient during the appointment. In some examples, the method 400 includes recording the association between the physical identifier 18 and the medical item in a memory, database, electronic health record system, treatment management system, pharmacy system, and/or another local and/or remote data storage system. In some examples, recording the association may facilitate maintenance of a chain-of-custody linkage between the patient encounter, the physical identifier 18, the unique identifier 24, and the medical item during one or more stages of the clinical workflow.

In some examples, associating the physical identifier 18 with the medical item includes physically attaching, positioning, coupling, and/or otherwise associating the physical identifier 18 with the medical item. As examples, the physical identifier 18 may be adhered to the medical item as a label or sticker, attached to the medical item as a tag, printed directly on packaging associated with the medical item, and/or otherwise physically associated with the medical item. In some examples, the physical identifier 18 may remain associated with the medical item during preparation, dispensing, transport, storage, review, verification, and/or administration of the appointment-specific treatment. As an example, the physical identifier 18 is a sticker adhered to an IV bag in the example in FIG. 5.

In some examples, associating the physical identifier 18 with the medical item includes associating the unique identifier 24 and/or appointment-specific treatment information with the medical item. In some examples, the physical identifier 18 associated with the medical item may facilitate maintenance of an association between the patient and the appointment-specific treatment information during one or more stages of the clinical workflow. In some examples, the physical identifier 18 associated with the medical item may facilitate closed-loop verification of the patient identification and the appointment-specific treatment information prior to administration of the appointment-specific treatment.

In some examples, the method 400 includes scanning a machine-readable code 26 associated with the physical identifier 18 after association of the physical identifier 18 with the medical item. In some examples, the method 400 includes retrieving appointment-specific treatment information associated with the patient and comparing the retrieved appointment-specific treatment information to treatment information associated with the medical item prior to administration of the appointment-specific treatment. In some examples, the method 400 includes recording, in response to verifying, a verification event associated with the patient identification and the appointment-specific treatment information. In some examples, recording the verification event includes storing information associated with verification of the patient identification and/or verification of the appointment-specific treatment information, e.g., on the external data source 22 as described above, as facilitated by the linkage between the physical identifier 18 and a corresponding digital treatment record associated with the patient and the appointment through the unique identifier 24.

With reference to block 425, the method 400 includes administering treatment after verification in blocks 410 and 415 and association with a medical item in block 420. In some examples, the method 400 includes administering the appointment-specific treatment to the patient in response to verification of the patient identification and/or the appointment-specific treatment information. In some examples, the method 400 includes administering the appointment-specific treatment after determining correspondence between the patient identification, the appointment-specific treatment information, the physical identifier 18, and/or the medical item associated with the appointment-specific treatment.

In some examples, the method 400 includes determining that a mismatch exists between the patient identification, the appointment-specific treatment information, the physical identifier 18, the medical item, and/or stored treatment information prior to administration of the appointment-specific treatment. In some examples, the method 400 includes generating an alert, notification, warning message, alarm, and/or other indication in response to determining the mismatch. In some examples, with reference to block 430, the method 400 may prevent, suspend, delay, and/or halt administration of the appointment-specific treatment in response to determining the mismatch.

In some examples, administering the treatment includes administering a medication, infusion, biologic treatment, injectable treatment, chemotherapy treatment, procedure, diagnostic treatment, and/or another treatment associated with the appointment-specific treatment information. In some examples, administering the treatment includes administering the treatment according to medication information, treatment information, dose information, administration route information, infusion information, physician information, scheduling information, and/or other appointment-specific treatment information associated with the patient and the appointment.

In some examples, the method 400 includes utilizing the physical identifier 18 during administration of the appointment-specific treatment. As examples, the physical identifier 18 may remain associated with the patient, the medical item, and/or the appointment-specific treatment during administration of the treatment. In some examples, the method 400 includes scanning a machine-readable code 26 associated with the physical identifier 18 prior to and/or during administration of the appointment-specific treatment. In some examples, the method 400 includes retrieving appointment-specific treatment information associated with the patient in response to the machine-readable scan and comparing the retrieved appointment-specific treatment information to treatment information associated with the treatment being administered to the patient.

Administration of the treatment after verification in blocks 410 and 415 facilitates maintenance of an association between the patient and the appointment-specific treatment information during the clinical workflow. In some examples, the verification and administration operations may facilitate closed-loop verification of the patient identification and the appointment-specific treatment information during administration of the appointment-specific treatment.

In some examples, the method 400 includes updating one or more external data sources 22 in response to one or more stages of the clinical workflow associated with the appointment-specific treatment. As examples, the method 400 may include updating an electronic health record system, pharmacy system, treatment management system, scheduling system, billing system, cloud computing platform, remote server, and/or another external data source 22 with information associated with preparation, dispensing, transport, storage, verification, administration, completion, interruption, suspension, and/or termination of the appointment-specific treatment associated with the patient and the appointment.

In some examples, the method 400 includes transmitting information associated with administration of the appointment-specific treatment to the external data source 22 after administration of the appointment-specific treatment to the patient. As examples, the transmitted information may include treatment status information, administration time information, dose administration information, administration route information, medical provider information, workflow information, verification event information, medical item information, completion information, and/or other information associated with treatment of the patient during the appointment.

Updating the external data source 22 may be facilitated by the linkage between the physical identifier 18 and a corresponding digital treatment record associated with the patient and the appointment through the unique identifier 24, as described above. In some examples, the method 400 includes utilizing the unique identifier 24 obtained from the physical identifier 18 and/or a machine-readable code 26 associated with the physical identifier 18 to identify, retrieve, update, and/or modify the corresponding digital treatment record associated with the patient and the appointment. In some examples, updating the external data source 22 may facilitate maintenance of continuity of appointment-specific treatment information during one or more stages of the clinical workflow and after completion of administration of the appointment-specific treatment.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A system comprising:
a user interface accessible by a patient during a patient check-in associated with an appointment;
a data interface configured to communicate with an external data source to obtain appointment-specific treatment information;
a computer having a processor and memory storing instructions executable by the processor to:
receive patient identification from the user interface during the patient check-in for the appointment;
transmit, via the data interface, a request for appointment-specific treatment information associated with the patient based on the patient identification;
receive, via the data interface, the appointment-specific treatment information;
generate a unique identifier based on the patient identification and the appointment-specific treatment information; and
generate, in response to receiving the appointment-specific treatment information during the patient check-in, a physical identifier including the patient identification, the appointment-specific treatment information, and the unique identifier.

2. The system as set forth in claim 1, wherein the appointment-specific treatment information includes at least one of a medication, a dose, or an administration route.

3. The system as set forth in claim 1, wherein the physical identifier includes human-readable representation of the appointment-specific treatment information.

4. The system as set forth in claim 1, wherein the physical identifier includes a machine-readable code encoding at least the unique identifier.

5. The system as set forth in claim 4, wherein the machine-readable code encodes at least the unique identifier and at least a portion of the appointment-specific treatment information.

6. The system as set forth in claim 4, wherein the unique identifier is based on a combination of information obtained from the plurality of external data sources.

7. The system as set forth in claim 1, wherein the data interface is configured to communicate with a plurality of external data sources.

8. The system as set forth in claim 6, wherein the plurality of external data sources includes at least two of an electronic health record system, a scheduling system, an insurance system, or a pharmacy system.

9. The system as set forth in claim 1, wherein the unique identifier is valid only for the appointment associated with the patient check-in.

10. The system as set forth in claim 1, wherein the physical identifier is configured to associate the patient with the appointment-specific treatment information within a clinical workflow.

11. The system as set forth in claim 1, further comprising a printer configured to print the patient identification, the appointment-specific treatment information, and the unique identifier on the physical identifier.

12. A method comprising:
receiving patient identification via a user interface during a patient check-in associated with the appointment;
transmitting, via a data interface, a request for appointment-specific treatment information associated with the patient based on the patient identification;
receiving, via the data interface, the appointment-specific treatment information;
generating a unique identifier based on the patient identification and the appointment-specific treatment information; and
generating, in response to receiving the appointment-specific treatment information during the patient check-in, a physical identifier including the patient identification, the appointment-specific treatment information, and the unique identifier.

13. The method as set forth in claim 12, further comprising verifying, at a point of treatment, that the patient identification corresponds to the physical identifier prior to administration of the appointment-specific treatment.

14. The method as set forth in claim 12, further comprising verifying, at a point of treatment, that the appointment-specific treatment information corresponds to a treatment to be administered to the patient prior to administration of the appointment-specific treatment.

15. The method as set forth in claim 12, further comprising associating the physical identifier with a medical item to be used in administration of the appointment-specific treatment.

16. The method as set forth in claim 12, wherein the appointment-specific treatment information includes at least one of a medication, a dose, or an administration route.

17. The method as set forth in claim 12, wherein the physical identifier includes human-readable representation of the appointment-specific treatment information.

18. The method as set forth in claim 12, wherein the physical identifier includes a machine-readable code encoding at least the unique identifier.

19. The method as set forth in claim 15, wherein the machine-readable code encodes at least the unique identifier and at least a portion of the appointment-specific treatment information.

20. The method as set forth in claim 12, wherein:
receiving appointment-specific treatment information includes receiving the appointment-specific treatment information includes receiving appointment-specific treatment information from a plurality of external data sources; and
generating the physical identifier includes combining appointment-specific treatment information obtained from the plurality of external data sources.
